(19) United States
(12) Patent Application Publication
(10) Pub. No.: US 2012/0177762 A1
(43) Pub. Date: Jul. 12, 2012

(54) INHIBITION OF EXOPROTEINS USING BACTERIA OF THE LACTOBACILLUS GENUS

(76) Inventor: Petr Kotal, Praha (CZ)

(21) Appl. No.: 13/382,414

(22) PCT Filed: Jul. 8, 2010
(86) PCT No.: PCT/CZ10/00075

§ 371 Date: Mar. 28, 2012

(30) Foreign Application Priority Data
Jul. 9, 2009 (CZ) PV 2009-444

(51) Int. Cl. A61K 35/74; A61P 31/04; C12N 1/20

(52) U.S. Cl. 424/780; 435/252.9

(57) ABSTRACT

Use of viable bacteria of the Lactobacillus genus or their cell extracts for the production of an absorption sanitary product for inhibition of growth of Gram-positive bacteria and of production of their exoproteins. Related absorption sanitary products.

### TECHNICAL FIELD

The invention deals with the use of bacteria of the Lactobacillus genus for the production of sanitary goods for inhibition of growth of Gram-positive bacteria and for inhibition of production of their exoproteins.

### BACKGROUND ART

Disposable absorption products for absorption of body fluids represent widely used sanitary goods. One of the most frequently used types is menstrual tampons.

It is known that in the urogenital microflora of healthy women before and after the menopause bacteria of the Lactobacillus genus prevail and that these lactobacilli have the ability to control growth and reduce pathogenicity of many uropathogens. They can also support faster recovery of natural microflora after antibiotic treatment.

Use of lactobacilli for impregnation of sanitary aids, e.g. menstrual tampons and napkins for prevention of urogenital infections is known from the document WO 92/13577. A procedure of production of absorption sanitary products containing bacteria producing lactic acid is described in EP patent 1 322 346; some bacteria producing lactic acid that can be used for this purpose are mentioned in EP patent 1 427 808.

The normal environment of the urogenital tract is inconvenient for some species of pathogenic bacteria such as Staphylococcus aureus. This preventive effect is very desirable as Staphylococcus aureus is able to produce and excrete to its surroundings various exoproteins and among them exotoxins, e.g. the toxic shock syndrome (TSS) toxin.

The toxic shock syndrome is generally known in connection with the use of menstrual tampons. The etiological agent is most frequently Staphylococcus aureus, producing the TSST-1 toxin, or staphylococcal enterotoxin, or both at the same time. As a separate nosological unit TSS was first described by J. Todd et al. in 1978 for 7 children at the age of 8-17 years, whose common symptoms were high temperature (39-41° C.), headaches, bemusement, hyperaemia of conjunctivae, non-itching scarlatiniform exanthema and hypotension. In 1980 this symptom complex was described in the U.S. in connection with the use of high-absorption vaginal tampons. In more than 90% of strains isolated from samples taken from women that had fallen ill a new toxin was identified, called the toxic shock syndrome toxin-1 (TSST-1) (Issa N., Thompson R., Staphylococcal toxic shock syndrome Postgraduate med. 2001; 10). TSS is a disease caused by superantigens. This is a group of proteins produced by bacteria that are able to activate the immune system without previous processing by the antigen presenting cell. The toxic shock syndrome toxin-1 (TSST-1) also belongs to these antigens. The “super” prefix characterizes their extraordinary characteristic: while common peptide antigens are able to induce a response in one T lymphocyte out of 100,000 to 1,000,000 T lymphocytes, the above mentioned high-potency superantigens can do the same with 5 to 30% of all T lymphocytes at the same time. This is because it circumvents the common way of processing and presentation of the antigen by the antigen presenting cell. This way the subpopulations of CD4 and CD8 T lymphocytes are stimulated and cytokins (TNFα, IL2, IL6) are released in a massive manner (Krejsek J, Kopecky O. Klinicka imunologie 2004; 141-142), which cytokins are responsible for the clinical symptoms of TSS, especially the capillary leak syndrome with redistribution of body fluids, hypoalbuminemia, oedemas and shock picture. What is important from the pathogenetic point of view is that the entire clinical picture is toxaemia, i.e. is caused by toxins penetrating from the primary focal source through mucous membranes or tissues to the blood circulation.

The toxic shock syndrome is mentioned in the document WO 2006/033950 generally as a syndrome related to the presence of Staphylococcus aureus and exotoxins. The general part mentions that prevention of TSS consists in increased hygiene in the menstrual period, use of antibiotics or bacteriocidal agents and reduction of pH by administration of organic acids. It does not mention that prevention could be ensured by administration of a lactobacillus. The use of lactobacilli which the document claims relates to maintaining and restoring urogenital microflora in humans.

TSS does not belong to urogenital infections since, as mentioned above, it is a systemic immune failure, which may be caused by the presence of Staphylococcus aureus both in the blood circulation and in the urogenital tract. TSS is not caused by the bacteria itself but by the toxic effects of the associated exotoxin. It is a very dangerous disease with fast progression that may even be fatal.

Several documents deal with prevention of TSS. U.S. Pat. No. 7,105,177 describes the use of derivatives of hop acids for impregnation of children's diapers and wet wipes as prevention of the toxic shock syndrome in infants. U.S. Pat. No. 7,056,891 relates to inhibition of production of exoproteins from Gram-positive bacteria by means of polyalkylglycoside compounds impregnated in menstrual tampons. Use of various chemical substances in sanitary products for TSS prevention is also described in the following documents: EP 395 099, WO99/12505, US 2003/0100871 and US 2007/0190121.

However, chemical substances require registration and compliance with strict regulative requirements, which makes their use in consumer goods such as menstrual tampons complicated.

### DISCLOSURE OF INVENTION

A probiotic agent has been found out, which is effective in inhibition of Gram-positive bacteria, especially the Staphylococcus genus, and can be used for impregnation of the variety of sanitary products. Lactobacteria have been found out to be effective in a new indication area, namely for prevention of the toxic shock syndrome by inhibiting the growth of Gram-positive bacteria and production of their exoproteins but as well for healing and prevention of vaginoses.

The present invention consists in use of viable bacteria of the Lactobacillus genus, or their cell extracts, for the production of an absorption sanitary product for inhibition of growth of Gram-positive bacteria and inhibition of production of their exoproteins. The use of the above mentioned bacteria or their cell extracts for inhibition of Staphylococcus aureus bacteria is preferable. The above mentioned bacteria can preferably be used for the production of sanitary products for prevention of the toxic shock syndrome.

Preferably, viable bacteria like Lactobacillus casei, Lactobacillus curvatus, Lactobacillus paracasei and/or Lactobacillus acidophilus, or their cell extracts, can be used.

Bacteria strains Lactobacillus casei 2750, Lactobacillus casei 2775, Lactobacillus curvatus 2775, Lactobacillus paracasei ST68, Lactobacillus paracasei 171R2, Lactobacillus paracasei SF1, Lactobacillus acidophilus CH5 and Lactobacillus Shirota have proved to be particularly preferable.

Another object of the invention is an absorption sanitary product for inhibition of growth of Gram-positive bacteria and of production of their exoproteins, which contains viable Lactobacillus spp bacteria, or their cells extracts. Preferably, the sanitary product is a menstrual tampon. Preferable sanitary products also include menstrual pads, napkins and panty liners.

The production of absorption sanitary products can be accomplished by the process that is described in EP patent 1 322 346. This procedure makes sure that the bacteria will remain viable during the processing and will also be viable in the final product.

The method of the invention has an advantage of being probiotic prevention of the toxic shock syndrome by means of consumer sanitary products. Lactobacillus is a probiotic agent providing the organism with natural protection without the use of foreign chemical substances.

### BRIEF DESCRIPTION OF DRAWINGS

FIG. 1 illustrates the antibacterial activity of the Lactobacillus casei 2750 strain against Staphylococcus aureus CCM 3953: 2750—living cells, A—supernatant after neutralization, B—supernatant after neutralization and treatment with catalase, C—supernatant after neutralization, treatment with catalase and thermal inactivation.

FIG. 2 shows the antibacterial activity of the Lactobacillus curvatus 2775 strain against Staphylococcus aureus CCM 3953: 2775—living cells, A—supernatant after neutralization, B—supernatant after neutralization and treatment with catalase, C—supernatant after neutralization, treatment with catalase and thermal inactivation.

### EXAMPLES

Tests of inhibition of Staphylococcus aureus by lactobacilli have been carried out.

Material and Methods:

List of Used Strains:
Staphylococcus aureus CCM 3953 (=ATCC 25923, Clinical isolate, international standard reference strain for antibacterial disk-susceptibility-testing; control strain for media testing, coagulase, Slidex Staph Plus, beta-LACTAMtest)
Lactobacillus paracasei ST68 (ICT Prague)
Lactobacillus paracasei 171R2 (Department of Food Science, University of Copenhagen, DK)
Lactobacillus acidophilus CH5 (commercial strain of the Chr. Hansen Company, DK)
Lactobacillus paracasei SF1 (University of Nebraska, Lincoln, USA)
Lactobacillus Shirota (Dairy Institute of the Agricultural Faculty of Perugia, IT)
Lactobacillus curvatus 2775 (Dairy Institute of the Agricultural Faculty of Perugia, IT)
Lactobacillus casei 2750 (Dairy Institute of the Agricultural Faculty of Perugia, IT)

Strains deposited in the collection of the Department of Dairy and Fat Technology of the Institute of Chemical Technology (ICT), Prague were used; the strains coming from foreign collections are donations obtained on the basis of expert contacts and cooperation. The strains in the above mentioned collection are stored in the standard way and are available to the public, especially for the purposes of science and research. The strains were used for the tests with consent of their owner and the depositing entity.

Cultivation of the Used Strains:
Staphylococcus aureus CCM 3953—BHI broth, MRS broth, 37° C./24 h
Lactobacillus spp.—MRS broth, 37° C./24h

Preparation of the Supernatant
Supernatant A: The freshly grown strain with antibacterial activity was centrifuged (3680 g, 4° C., 15 min) and its pH was adjusted to the value of 6-6.5 by usage of NaOH (1 and 10 w/w % solution) (FRANZ, C.M.A.P.; SCHILLINGER, U.; HOLZAPFEL, W. H. Production and characterization of enterocin 900, a bacteriocin produced by Enterococcus faecium BFE 900 from black olives. Int. J. Food Microbiol., 1996, 29, 255-270).
Supernatant B: Catalase was added to supernatant A (the final concentration of catalase was 1 mg·ml⁻¹) and incubation at the temperature of 37° C./2 hours followed.
Supernatant C: Supernatant B with catalase was heated (90° C./10 min) after the incubation to inactivate the enzyme before testing of the residual antibacterial activity, (KANG, J. H.; LEE, M. S. Characterization of a bacteriocin produced by Enterococcus faecium GM-1 isolated from an infant. J. Appl. Microbiol., 2005, 98, 1169-1176).

Testing the Antibacterial Activity with the Agar Pinhole Method

The antibacterial activity was tested with the use of the agar well diffusion assay. A suspension of 1 vol. % of an active culture of Staphylococcus aureus, diluted to A_{600 nm}=0.7-0.8 in 30 ml of MRS agar (pH 6.0), was prepared and poured onto a Petri dish (9 cm diameter). The dishes were then dried at the room temperature for 2 hours. A cork borer was used for making a well (7 mm) in the pre-dried dishes into which 50 μl of the production culture or supernatant were dosed. The dishes were incubated at the temperature of 37° C. for 24 hours and the occurrence of inhibition zones, which indicated antibacterial activity, was observed (DAVE, R. I.; SHAH, N. P. Characteristics of bacteriocin produced by Lactobacillus acidophilus LA-1. Int. Dairy J., 1997, 7, 707-715.).

### RESULTS

Antibacterial activity of 7 lactobacilli strains (Lactobacillus paracasei ST68, Lactobacillus paracasei 171R2, Lactobacillus paracasei SF1, Lactobacillus acidophilus CH5, Lactobacillus Shirota, Lactobacillus curvatus 2775 and Lactobacillus casei 2750) against Staphylococcus aureus CCM 3953 was tested using the agar well diffusion assay. If the diameter of inhibition zones (after deduction of the cork borer diameter, i.e. 7 mm) was larger than 2 mm, these zones were considered as a positive result.

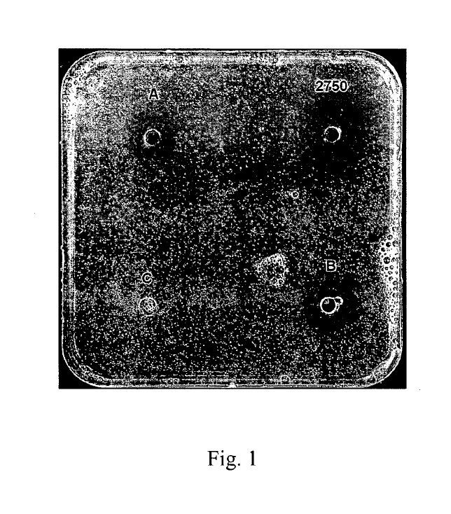

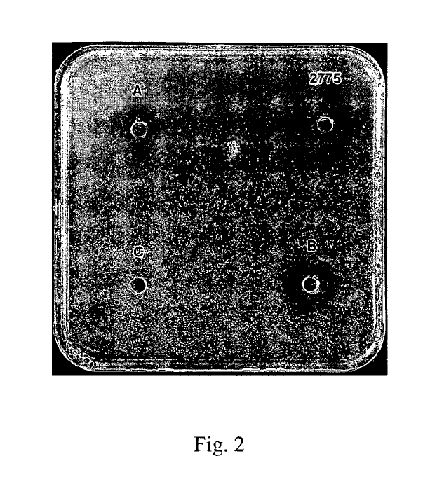

Lactobacillus acidophilus CH5 only inhibited the Staphylococcus aureus CCM 3953 indicator strain during the testing of living cells, i.e. it was only the production of acids that was responsible for the activity. In the Lactobacillus paracasei 171R2 strain activity was clearly demonstrated for living cells, in the supernatant after neutralization the diameter of the inhibition zone was 9 mm, which cannot be considered conclusive, and after treatment of the supernatant with catalase activity was no longer detected. This means that mainly the production of acids participated in the activity. In the other five strains activity was observed in supernatants treated with catalase as well. So their activity was probably caused by production of antibacterial substances; in the case of the SF1 and Shirota strains a decrease of the inhibition zone size in supernatants treated with catalase was observed, which means that hydrogen peroxide may also have participated in the activity. These antibacterial substances were thermolabile as after heat treatment no activity was observed in the case of any of the tested strains (see Table I, FIG. 1, FIG. 2).

TABLE I
Antibacterial activity of lactobacilli against
Staphylococcus aureus CCM3953

| | Inhibition zone diameter* [mm] | | | |
|---|---|---|---|---|
| | living | supernatant | supernatant | supernatant |
| Tested strains | cells | A | B | C |
| Lactobacillus | 22 | 13 | 13 | 0 |
| paracasei ST68 | | | | |
| Lactobacillus | 20 | 9 | 0 | 0 |
| paracasei 171R2 | | | | |
| Lactobacillus | 24 | 15 | 12 | 0 |
| paracasei SF1 | | | | |
| Lactobacillus | 15 | 0 | 0 | 0 |
| acidophilus CH5 | | | | |
| Lactobacillus Shirota | 22 | 15 | 11 | 0 |
| Lactobacillus curvatus | 25 | 16 | 16 | 0 |
| 2775 | | | | |
| Lactobacillus casei | 23 | 15 | 14 | 0 |
| 2750 | | | | |

*diameter of the inhibition zone without deduction of the diameter of the cork borer, i.e. 7 mm, A - supernatant after neutralization, B - supernatant after neutralization and catalase treatment, C - supernatant after neutralization, catalase treatment and heat treatment.

### CONCLUSION

Altogether 7 strains of Lactobacillus spp. were tested for antibacterial activity against the Staphylococcus aureus CCM 3953 indicator strain. Antibacterial activity against the indicator strain caused by production of an antibacterial substance other than an organic acid was observed in the case of 5 lactobacilli. In the remaining 2 lactobacilli it was only the production of acids that was responsible for the inhibition.

1. Use of viable bacteria of the Lactobacillus genus or their cell extracts for the production of an absorption sanitary product for inhibition of growth of Gram-positive bacteria and of production of their exoproteins.

2. The use according to claim 1, wherein the Gram-positive bacterium is Staphylococcus aureus.

3. The use according to claim 1 for sanitary products for prevention of the toxic shock syndrome.

4. The use according to claim 1, wherein the viable bacteria are selected from the group consisting of Lactobacillus casei, Lactobacillus curvatus, Lactobacillus paracasei and/or Lactobacillus acidophilus, or their cell extracts.

5. The use according to claim 4, wherein the viable bacteria are of the Lactobacillus casei 2750 strain, or their cell extract.

6. The use according to claim 4, wherein the viable bacteria are of the Lactobacillus curvatus 2775 strain, or their cell extract.

7. The use according to claim 4, wherein the viable bacteria are of the Lactobacillus paracasei ST68 strain, or their cell extract.

8. The use according to claim 4, wherein the viable bacteria are of the Lactobacillus paracasei SF1 strain, or their cell extract.

9. The use according to claim 4, wherein the viable bacteria are of the Lactobacillus Shirota strain, or their cell extract.

10. An absorption sanitary product for inhibition of growth of Gram-positive bacteria and production of their exoproteins, characterized in that it comprises viable bacteria of the Lactobacillus spp. genus or their cell extract.

11. The product according to claim 10 for prevention of the toxic shock syndrome.

12. The product according to claim 10, which is a menstrual tampon.

13. The product according to claim 10, which is a menstrual pad or napkin.

14. The product according to claim 10, which is a panty liner.
